(19) United States
(12) Patent Application Publication
(10) Pub. No.: US 2023/0303961 A1
(43) Pub. Date: Sep. 28, 2023

(54) SAMPLE MEASUREMENT APPARATUS, SAMPLE MEASUREMENT SYSTEM, AND ARTIFICIAL INSEMINATION APPARATUS

(71) Applicants: Hitachi, Ltd., Tokyo (JP); National Institutes for Quantum Science and Technology, Chiba-shi (JP)

(72) Inventors: Masanari KOGUCHI, Tokyo (JP); Takeshi NAKAYAMA, Tokyo (JP); Ryuji IGARASHI, Chiba (JP); Kiichi KAMINAGA, Chiba (JP); Satoshi TSUKAMOTO, Chiba (JP)

(73) Assignees: Hitachi, Ltd., Tokyo (JP); National Institutes for Quantum Science and Technology, Chiba-shi (JP)

(21) Appl. No.: 18/179,557

(22) Filed: Mar. 7, 2023

(30) Foreign Application Priority Data
Mar. 23, 2022 (JP) 2022-046774

(51) Int. Cl. C12M 1/34; C12M 1/12; C12M 1/42; G01N 21/64

(52) U.S. Cl. CPC C12M 41/46; C12M 25/10; C12M 35/00; G01N 21/6428

(57) ABSTRACT

An object of the invention is to make it possible to provide a mechanism that applies an external stimulus and to measure structural and electromagnetic changes of a cell due to the external stimulus with high sensitivity by an NV center. There is provided an environment control mechanism configured to change a state of a sample by applying an external stimulus to the sample.

### BACKGROUND OF THE INVENTION

### 1. Field of the Invention

The present invention relates to a sample measurement apparatus, a sample measurement system, and an artificial insemination apparatus.

### 2. Description of Related Art

As research and development of new materials for electronics to which regenerative medicine and biological functions are applied become active, visualization of biological material functions is a problem. That is, in a case of a cell, a process monitor of differentiation and induction and a technique of quantitatively measuring signal propagation between an ion channel and an organelle are required.

Meter measurements of processes and functions illustrated here should be considered as various physical quantities such as an electric field, a magnetic field, a temperature, pH, an ion flow, and light emission accompanying a reaction. In this regard, techniques are widely used to, for example, trace a reaction process by using a fluorescent dye that is likely to accompany a specific protein as a label, or to trace the reaction process by extracting a specific organelle in a cell by a centrifugal separation technique or the like, and then analyzing genes. However, there are problems with spatial resolution being at a submicrometer level, which is defined by a wavelength of light, and high degree of invasiveness, such as destruction of the cell.

In recent years, a diamond material including an NV center (nitrogen-vacancy pair) has attracted attention for such a biofunctional measurement because the diamond material has high sensitivity to a minute electromagnetic field and a temperature, has high spatial resolution because a size of a sensor is at an atomic level, and is excellent in biocompatibility because the material is a carbon material.

A measurement technique using the NV center includes (1) a method of using a nanoparticle including the NV center and (2) a method of a scanning probe microscope using a diamond probe including the NV center (for example, see JP2015-529328A (Japanese Patent No. 6117926)).

However, in the measurement using the nanoparticle including the NV center in the method (1), only a vicinity of a portion where the nanoparticle is present can be measured, and position controllability is not high. When the nanoparticles are aggregated, there is a problem that the spatial resolution deteriorates accordingly.

### SUMMARY OF THE INVENTION

In the above method (2) of the scanning probe microscope using the probe including the NV center, high spatial resolution can be expected, and there is no problem with physical quantities, such as a magnetic field and an electric field, that can be measured in a non-contact manner with respect to a sample.

However, in JP2015-529328A (Japanese Patent No. 6117926), since there is no mechanism that applies an external stimulus to a cell, it is not possible to evaluate cell changes that is dynamics such as before and after insemination, before and after drug stimulation, and before and after neural firing signal input.

In a case of an apparatus configuration illustrated in JP2015-529328A (Japanese Patent No. 6117926), that is, in the case of the configuration in which a laser light that excites the NV center is emitted from the probe side when the sample is scanned using a probe, there is a problem that a space between a lens and the sample can be usually secured only by several mm due to a characteristic of the lens that converges the laser light, and it is difficult to provide the above mechanism that applies the external stimulus.

An object of the invention is to make it possible to provide a mechanism that applies an external stimulus and to measure, by an NV center, structural and electromagnetic changes of a cell due to the external stimulus with high sensitivity.

A sample measurement apparatus according to an aspect of the invention measures a state of a sample using a probe made of diamond or silicon carbide including a nitrogen-vacancy pair. The sample measurement apparatus includes an environment control mechanism provided in a vicinity of the sample, and the environmental control mechanism changes the state of the sample by applying an external stimulus to the sample.

An artificial insemination apparatus according to an aspect of the invention measures a reaction of a cell using a probe made of diamond or silicon carbide including a nitrogen-vacancy pair. The artificial insemination apparatus includes an environment control mechanism provided in the vicinity of the sample, and the environment control mechanism changes a state of the cell by applying the external stimulus to the cell.

According to an aspect of the invention, a mechanism that applies an external stimulus can be provided, and structural and electromagnetic changes of a cell due to the external stimulus can be measured with high sensitivity by an NV center.

### BRIEF DESCRIPTION OF THE DRAWINGS

FIG. 1A is a diagram illustrating a crystal structure of diamond including an NV center;

FIG. 1B is a diagram illustrating an optically detected magnetic resonance spectrum;

FIG. 1C is a diagram illustrating a state of electron energy;

FIG. 2 is a front view illustrating a basic configuration of an upright prober apparatus using diamond including an NV center as a probe according to a first embodiment;

FIG. 3 is a side view illustrating the basic configuration of the upright prober apparatus using the diamond including the NV center as the probe according to the first embodiment;

FIG. 4 is a front view illustrating a basic configuration of an inverted prober apparatus using diamond including an NV center as a probe according to a second embodiment;

FIGS. 5A to 5G are views illustrating a process of manufacturing a probe for a prober apparatus by a focused ion beam and a microsampling method according to a third embodiment;

FIG. 6 is a top view illustrating the basic configuration of the prober apparatus using the diamond including the NV center as the probe according to the first embodiment; and

FIG. 7 is a top view illustrating the basic configuration of the prober apparatus including a rotary table using the diamond including the NV center as the probe according to the first embodiment.

### DESCRIPTION OF EMBODIMENTS

Hereinafter, embodiments of the invention will be described in detail with reference to drawings. In all the drawings illustrating the embodiments, the same components are denoted by the same reference numerals in principle, and the repeated description thereof is omitted.

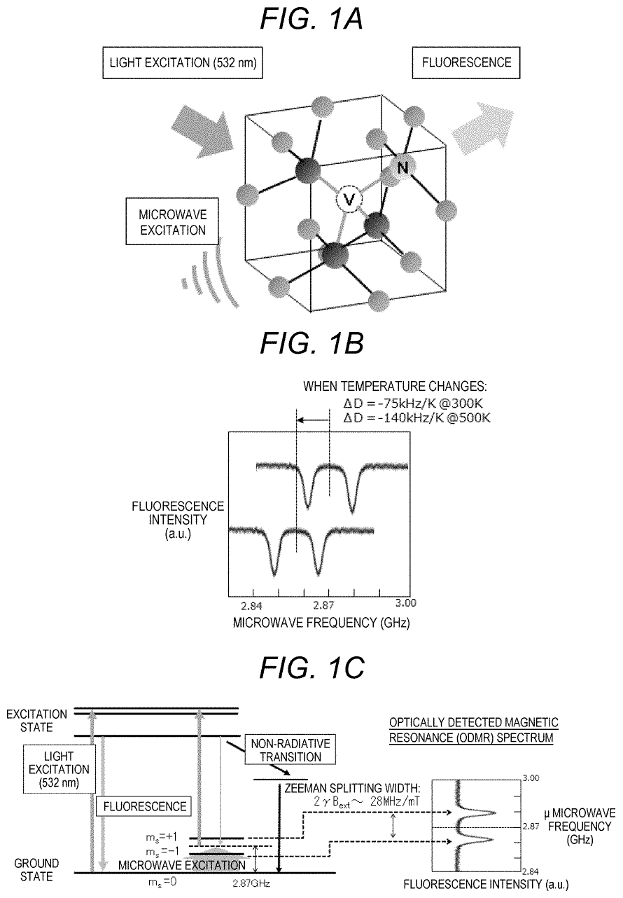

First, a general technique serving as a premise of the invention will be described with reference to FIG. 1A, FIG. 1B, and FIG. 1C.

First, a unit cell structure of diamond including an NV center is illustrated in FIG. 1A.

Generally, a constant number of vacancies (V) is introduced into a diamond substrate containing nitrogen (N) by electron beam irradiation or the like. Then, by performing high-temperature annealing, nitrogen (N) and vacancies (V) are rearranged at positions adjacent to each other in a <111> direction, and are stabilized in terms of energy. A luminescent center formed in the diamond is called an NV center due to an atomic structure thereof.

Such a crystal takes an electron energy state having characteristics illustrated in FIG. 1C. An NV pair usually captures one electron to form a monovalent NV, and the electron forms a spin triplet state. When green light having a wavelength of 532 nm is emitted in a state in which nothing is done, an electron excited from a state of ms=0 emits red fluorescence having a longer wavelength (about 550 nm to 800 nm), thereby relaxing the electron to the original state of ms=0. On the other hand, when this crystal is irradiated with a microwave around 2.87 GHz, the electron can be excited from the state of ms=0 to a state of ms=±1 by electron spin resonance. When the green light having the wavelength of 532 nm is emitted in the state of ms=±1, a part of the electrons is relaxed to the state of ms=0 through non-radiative transition.

In this case, the red fluorescence decreases accordingly. The state of ms=±1 is degenerate in the absence of a magnetic field, but splits into two levels due to Zeeman splitting in the presence of the magnetic field. By taking advantage of this feature, it is possible to accurately measure a resonance level by electron spin resonance (ESR) by sweeping the wavelength of the microwave that excites the electron from the state of ms=0 to the state of ms=±1. A Zeeman splitting width is proportional to the magnetic field sensed by the NV center, and the magnetic field can be measured based on the above two-level width.

That is, when the magnetic field is applied to the NV center, a resonance spectrum on a right side in FIG. 1C is obtained, and the applied magnetic field can be calculated based on an energy difference (here, frequency difference) between two peaks. For example, it is known that when a magnetic field of 1 gauss (0.1 mT) is applied to the NV center at room temperature, a peak interval is about 2.8 MHz. This spectrum is generally called an optically detected magnetic resonance (ODMR) spectrum.

It is known that in this ODMR spectrum, an energy (frequency) shift further occurs when there is a temperature change at an NV center position, and as illustrated in FIG. 1B, a shift amount of −75 kHz/K is obtained at room temperature 300K, and a shift amount of −140 kHz/K is obtained at 500K. Therefore, the temperature at which the NV center is placed can be measured based on the shift amount, and accuracy of the temperature is assumed to be 10 mK or less.

A measurement technique using the NV center may have an atomic level spatial resolution because a sensing portion thereof has an atomic level size as illustrated in FIG. 1A. However, since detection is executed using light, the light emitting point spreads over the wavelength of the light, that is, about several 100 nm. Accordingly, there are two methods to achieve high spatial resolution when measuring an electromagnetic field and a temperature of a sample by the NV center. The two methods are (1) a method of using a nanoparticle including the NV center and (2) a method of a scanning probe microscope using a diamond probe including the NV center.

Regarding the method (1) using the nanoparticle including the NV center, a carbon nanoparticle including the NV center is already commercially available and is contained in the sample to be measured. The red fluorescence illustrated in FIGS. 1B and 1C is detected by irradiating the carbon nanoparticle with the green laser light having the wavelength of 532 nm and the microwave. As for the spatial resolution, the nanoparticle size can be obtained when a position where the nanoparticle is placed can be accurately recognized. Here, it can be said that the above green laser light is optimal excitation light when the wavelength of the green laser light is 520 nm or more and 540 nm or less. On the other hand, as the laser light used for irradiation, for example, yellow-green laser light having a wavelength of 561 nm can also be applied.

Next, an outline of an apparatus configuration of the scanning probe microscope using the diamond probe including the NV center in the method (2) will be described. The apparatus configuration will be described based on a general scanning probe microscope (SPM).

That is, a sample is placed on a sample stage on an optical bench having a vibration isolation function, and the sample is scanned from above by the diamond probe including the NV center. A microwave antenna applied to the sample is provided in a vicinity of the sample.

On the other hand, emitted light from the green laser light having the wavelength of 532 nm that excites the electron is aligned, and the green laser light is transmitted through a transparent base material, and the probe is irradiated with the green laser light. During this process, the green laser light passes through an acousto-optic modulator (AOM). The AOM forms a stationary wave in the crystal by a vibration of a piezoelectric element, and uses the stationary wave as a diffraction grating. Since a grating width of the diffraction grating can be controlled by a vibration frequency applied to the crystal, an angle of the light bent through the diffraction grating can be freely changed. Here, instead of the above AOM, another modulator such as an electro-optic modulator (EOM) may be applied.

The NV center is provided at a tip of the probe, and the red fluorescence from the NV center passes through an optical path opposite to that of an incident laser light and is detected by an avalanche photodiode detector (APD detector) via a half mirror. The green laser light is guided to a beam profiler in order to adjust a beam intensity and a shape, and the red fluorescence is guided to a spectrometer in order to grasp a light emission characteristic of the red fluorescence.

A microwave power amplifier and the APD detector are connected to a control system device, and the probe microscope is connected to an SPM controller. Both of these devices are connected by communication, and are mechanisms that can control time sequences of microwave, sample stage control, and the like. These devices are provided in a dark room in order to avoid stray light from entering the detector.

As an electromagnetic field measurement method, high spatial resolution measurement of about several tens of nanometers is expected. The diamond probe including the NV center is formed of diamond together with the transparent base material.

In the measurement using the nanoparticle including the NV center in the method (1), only a vicinity of a portion where the nanoparticle is present can be measured, and position controllability is not high. When the nanoparticles are aggregated, there is a problem that the spatial resolution deteriorates accordingly. When the nanoparticle is introduced into a cell, it is extremely difficult to discharge the nanoparticle to an outside of the cell after evaluation and measurement, and thus it is difficult to use the cell selected for good or bad for artificial insemination or regenerative medicine.

In the method (2) of the scanning probe microscope using the probe including the NV center, the high spatial resolution can be expected, and there is no problem with physical quantities, such as a magnetic field and an electric field, that can be measured in a non-contact manner with respect to the sample.

As described above, in a microscope system using a diamond probe including a general NV center, it is possible to observe an electromagnetic field with high resolution by bringing the NV diamond probe into contact with the sample. However, no mechanism is provided to control an environment of the sample, and it is not possible to evaluate the state change of the sample during drug stimulation and insemination.

since it is necessary to bring a lens having a high light collection rate close to the sample in order to execute high-sensitivity fluorescence detection, a space around the sample is narrow, and it is difficult to provide a mechanism to control the environment.

As described above, since there is no mechanism that applies an external stimulus to a cell, it is not possible to evaluate cell changes that is dynamics such as before and after insemination, before and after drug stimulation, and before and after neural firing signal input.

That is, in a case of a configuration in which a laser light that excites the NV center is emitted from the probe side when the sample is scanned using a probe, there is a problem that a space between the lens and the sample can be usually secured only by several mm due to a characteristic of the lens that converges the laser light, and it is difficult to provide the above mechanism that applies the external stimulus.

According to the invention, a mechanism that applies external stimulus can be provided, and structural and electromagnetic changes of a cell due to the external stimulus can be measured with high sensitivity by the NV center.

Therefore, in the invention, an environment control mechanism that changes the state of the sample is provided in the vicinity of the sample. A space around a sample chamber is secured by providing a mechanism related to the laser light that excites the NV center or the microwave, the probe, and the environment control mechanism on opposite surfaces to each other with respect to the sample stage, and the environmental control mechanism having a wider range of applications can be provided.

Hereinafter, embodiments of the invention will be described with reference to the drawings. Embodiments of the invention relate to a probing apparatus that measures an electromagnetic field, a temperature, a pH value, a composition, and spin information of a soft material in a liquid such as a cell with high spatial resolution.

### First Embodiment

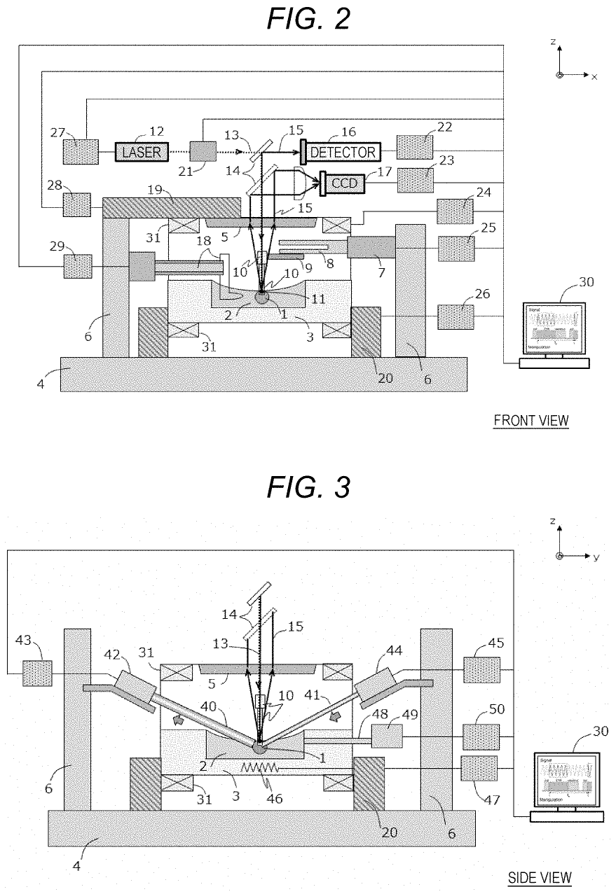

A sample measurement apparatus according to a first embodiment of the invention will be described with reference to FIGS. 2, 3, 6, and 7. The sample measurement apparatus according to the first embodiment is a prober apparatus using the diamond including the NV center as the probe.

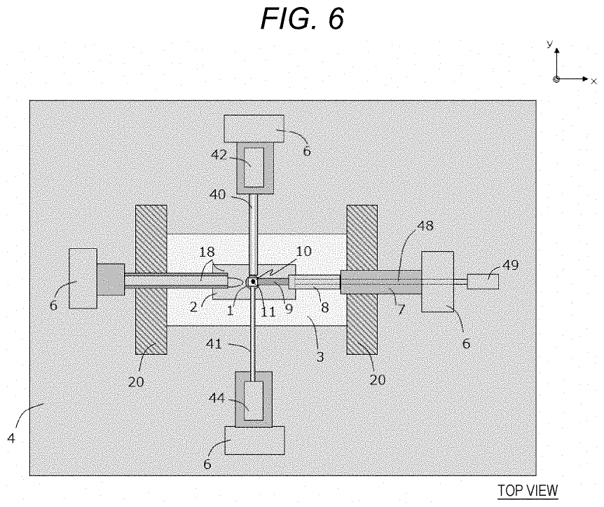

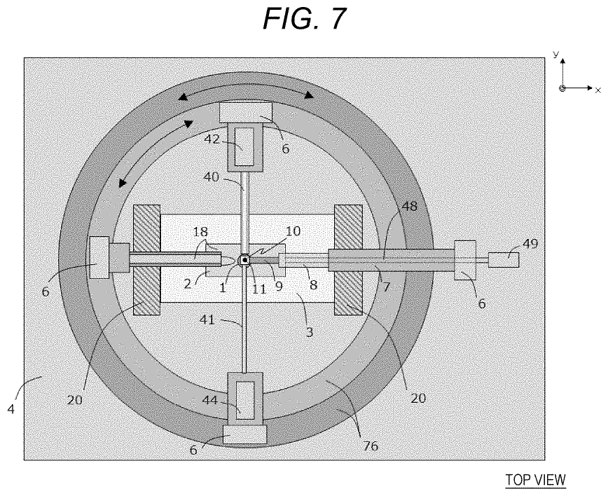

Here, FIG. 2 is a front view illustrating a basic configuration of an upright prober apparatus using the diamond including the NV center as the probe. FIG. 3 is a side view illustrating the basic configuration of the upright prober apparatus using the diamond including the NV center as the probe. FIG. 6 is a top view illustrating the basic configuration of the prober apparatus using the diamond including the NV center as the probe. FIG. 7 is a top view illustrating the basic configuration of the prober apparatus including a rotary table using the diamond including the NV center as the probe.

FIG. 2 illustrates the basic configuration of the prober apparatus using the diamond including the NV center as the probe. This configuration is called an upright configuration because the probe and the laser light approach the sample from the same surface.

In this apparatus configuration, a sample 1 is placed in a culture solution 2 on a sample stage 3. The entire apparatus to be described below is assembled on a vibration isolation table 4 in order to achieve measurement with high spatial resolution, and vibrations of members are prevented as much as possible. A diamond probe 10 including an NV center 11 formed at a tip of the diamond probe 10 is fixed on a probe base 9 made of glass or metal. The diamond probe 10 and the probe base 9 are fixed on a crystal oscillator 8 on a piezoelectric element 7.

Positions of the diamond probe 10 and the probe base 9 are controlled by a probe microscope control mechanism 25, and the diamond probe 10 is brought into contact with the sample 1. Although diamond is used as a material here, a similar quantum sensor material with a defect made of silicon carbide as a light emission point has also attracted attention, and it is possible to replace the probes made of these materials depending on applications.

In the measurement using the NV center 11 of the diamond, fluorescence is excited by the green laser light having the wavelength of 532 nm. Laser light 13 emitted from a laser light source 12 is emitted onto the sample 1 by a mirror 14 and a lens 5. As illustrated in FIG. 1, since red NV fluorescence 15 is emitted from the sample 1, the NV fluorescent light 15 is collected by the lens 5 and is focused on a detector 16 or a CCD camera 17 via the mirror 14.

In the former case, a fluorescence amount is detected as a current or the number of fluorescence pulses, and in the latter case, a fluorescence image on a surface of the sample 1 is captured. Here, in order to align a positional relation between the lens 5 and the laser light, a lens fine adjustment mechanism 19 using the piezoelectric element, a motor, or the like is used. The laser light achieves various quantum measurement protocols by a time sequence in which a microwave and a timing to be described below are matched.

Therefore, in order to form a short time pulse with a high time resolution, an acousto-optic modulator 21, that is, a device called the acousto-optic modulator (AOM) is widely used between the laser light source 12 and the sample 1. A laser light intensity and the like are adjusted by a laser controller 27. A detector controller 22 and a CCD camera controller 23 are used for operation control and signal processing of the detector 16 and the CCD camera 17, respectively.

Next, the microwave, which is another irradiation element, will be described. As described with reference to FIG. 1, it is necessary to irradiate the sample with a microwave of about 2.87 GHz while scanning the frequency. Since it is important for high-sensitivity measurement to bring a microwave generation source close to a distance of several tens of micrometers from the sample 1, a microwave antenna 18 capable of being close to the sample is provided. The frequency, the intensity, the timing, and the like of the microwave antenna 18 are controlled by a microwave controller 29.

A relative positional relation between the sample stage 3 and the diamond probe 10 is controlled by a sample stage fine movement mechanism 20. The sample stage fine movement mechanism 20 is a mechanism capable of coarse movement and fine movement in x, y, and z directions of the sample stage 3 illustrated in the drawings, and a drive source controlled by a control mechanism for the sample stage fine movement controller 26 is formed using a conductive motor and a piezoelectric element in combination.

In order to control an electron spin of the NV center 11, it is necessary to apply a stable external magnetic field. Here, a pair of air core Helmholtz coils 31 sandwich the sample 1, and an intensity and a timing of a magnetic field generated by a Helmholtz coil controller 24 are controlled. The entire system described above is controlled by a system control PC 30.

FIG. 3 is the side view of the upright apparatus.

As illustrated in FIG. 3, the first embodiment is characterized in that a sample environment control mechanism is disposed.

First, as illustrated in FIG. 2, since the sample 1 floats in the culture solution 2, a relative positional relation between the sample 1 and the diamond probe 10 is not determined only by this. This is solved by fixing the sample 1 by a sample suction tube 40 such that the sample 1 does not move in the culture solution 2. A position of the sample suction tube 40 is controlled by a sample suction tube position and pressure control mechanism 42 formed by a motor and a piezoelectric element.

Next, for example, in order to fix the cell, it is effective to make the sample suction tube 40 to a slightly negative pressure, and the sample suction tube position and pressure control mechanism 42 controlled by a sample suction controller 43 is also used to adjust the pressure in the sample suction tube 40 for suctioning, fixing, and releasing the cell.

Next, the injection tube 41 is provided in a form of being inserted into the sample 1. In an evaluation of a process of the artificial insemination, in an evaluation of the effects of a sperm and gene editing, in an evaluation of drug effects in a gene drug such as CRISPR-Cas9 and a drug discovery research, and in an evaluation of a drug solution, a neuronal firing mechanism and a cell behavior, an ionic solution such as calcium or potassium is injected into a target organelle inside or outside the cell. For this, a glass pipetle-shaped member having a tip diameter at a micrometer level is assumed. An injection tube position control mechanism 44 formed by the motor and the piezoelectric element is used to cause the injection tube 41 to approach and to be inserted into the target cell. An injection controller 45 is also used to control the amount of injection drug here.

A large number of biological samples are fairly sensitive to changes in states depending on an environmental temperature. Therefore, as the environment control mechanism, temperature adjustment of the sample 1 and flow rate control of the culture solution 2 are important. Here, a heater 46 whose temperature is adjusted by a heater adjustment controller 47 is embedded in the sample stage 3.

A flow path 48 is provided in the sample stage 3, and the culture solution 2 flows by a pump 49 controlled by a flow rate controller 50. Various controllers illustrated in this figure are controlled by a system control PC 30.

FIG. 6 illustrates the top view of the prober apparatus using the diamond including the NV center illustrated in FIG. 2 as the probe. In this figure, in particular, various members provided at a height from the vibration isolation table 4 to a lower surface of the lens 5 are drawn, and a positional relation of the mechanisms is illustrated.

Since a large number of mechanisms approach the sample 1, it is necessary to arrange the mechanisms such that the mechanisms do not interfere with one another. That is, in FIG. 6, the diamond probe 10 approaches the sample 1 from a right side, and the microwave antenna 18 approaches the sample 1 from an opposing left side.

In particular, since it is essential for efficient microwave irradiation to bring the microwave antenna 18 close to the NV center 11 present in the diamond probe 10 by a distance of several tens of microns, it is effective to bring the microwave antenna 18 close to the NV center 11 from an open side of the diamond probe 10.

On the other hand, since the sample 1 is in the culture solution 2, for a high-precision measurement, it is effective to perform measurement with the diamond probe 10 while fixing the sample 1 with the sample suction tube 40 and injecting the sperm or drug into or out of the cell with the injection tube 41.

Since the injection tube 41 and the diamond probe 10 particularly exert a force when inserted into the sample 1, it is effective for stably fixing the sample 1 to provide the sample suction tube 40 at a position facing the injection tube 41 and the diamond probe 10.

FIG. 6 illustrates a structure in which the sample suction tube 40 and the injection tube 41 face each other and approach the sample 1 from above and below. Similarly, a system of the flow path 48 and the pump 49 needs to be disposed in a manner of not interfering with these mechanisms.

FIG. 6 illustrates a configuration diagram in which the flow path 48 is drawn out to a right side in the same direction below the diamond probe 10, the crystal oscillator 8, and the piezoelectric element 7, as the flow path 48 is indicated by a dotted line. A direction of the flow path 48 may be changed in the sample stage 3, and the flow path 48 may be taken out from, for example, a direction of a lower side of the sample stage 3 in this figure.

FIG. 7 illustrates an example in which each mechanism is placed on the rotary table 76 such that a relative angle relation between mechanisms to be brought close to the sample 1 can be changed.

The rotary table 76 in this figure has an independent double rotary stage structure, and is provided on the vibration isolation table 4 in a form of surrounding the sample stage 3. In the example in this figure, a side structure 6 that supports the sample suction tube 40 and a side structure 6 that supports the microwave antenna 18 are placed on the rotary stage in an inner circumference. A side structure 6 that supports the injection tube 41 is placed on the rotary stage on an outer circumference. A side structure 6 that supports the diamond probe 10 is placed on the vibration isolation table 4.

The relative angle relation between the mechanisms can be freely set by a combination of the angles of the rotary stage on the inner and outer circumferences. In particular, in a case in which the diamond probe 10 needs to be inserted into the sample 1 with a strong force, the suction performed by the sample suction tube 40 needs to be strong, and in this case, it is effective that the diamond probe 10 and the sample suction tube 40 face each other with the sample 1 interposed therebetween. Therefore, by the rotary table 76, for example, it is possible to change the disposition such that the sample suction tube 40 can approach the sample 1 from the left, the microwave antenna 18 can approach the sample 1 from below, and the injection tube 41 can approach the sample 1 from above.

The rotary table 76 may not have a double structure as illustrated in FIG. 7. The rotary table 76 may have a structure that does not have a rotation stroke over one turn, but has a rotation stroke over, for example, half a turn or less. The rotary table 76 may also have a structure that functions to move the side structure 6 in one direction and that retracts each mechanism as an unused mechanism when the mechanism is not in use.

As described above, in the first embodiment, the environment control mechanism is provided in the vicinity of the sample 1 in the sample measurement apparatus that measures the state of the sample 1 using the probe made of diamond or silicon carbide including the nitrogen-vacancy pair (NV center 11). The environment control mechanism changes the state of the sample 1 by applying the external stimulus to the sample 1.

In the first embodiment, an environment control mechanism is provided in the vicinity of the sample 1 in an artificial insemination apparatus that measures a reaction of a cell using the probe made of diamond or silicon carbide including the nitrogen-vacancy pair (NV center 11). The environment control mechanism changes the state of the cell by applying the external stimulus to the cell.

The environment control mechanism includes the sample suction tube 40 that determines a relative positional relation between the sample 1 floating in a solution (culture solution 2) and the probe and that fixes the sample 1 such that the sample 1 does not move in the solution.

The environment control mechanism includes the injection tube 41 that is inserted into the sample 1 and that injects a substance into the sample.

The environment control mechanism includes the sample suction tube 40 that determines the relative positional relation between the sample 1 floating in the solution and the probe and that fixes the sample 1 such that the sample 1 does not move in the solution, the injection tube 41 that is inserted into the sample and that injects a substance into the sample. The sample suction tube 40 and the injection tube 41 face each other at a predetermined angle with respect to the nitrogen-vacancy pair.

The environment control mechanism includes the heater 46 that is disposed inside the sample stage 3 and that adjusts the temperature of the sample, the flow path 48 that is disposed inside the sample stage 3 and that controls the flow rate of the solution.

According to the first embodiment, it is possible to cause a cell culture environment to be constant, reduce the deterioration of the culture solution, and execute evaluation measurement that is more stable for a longer period of time and that is close to an environment in an animal body.

### Second Embodiment

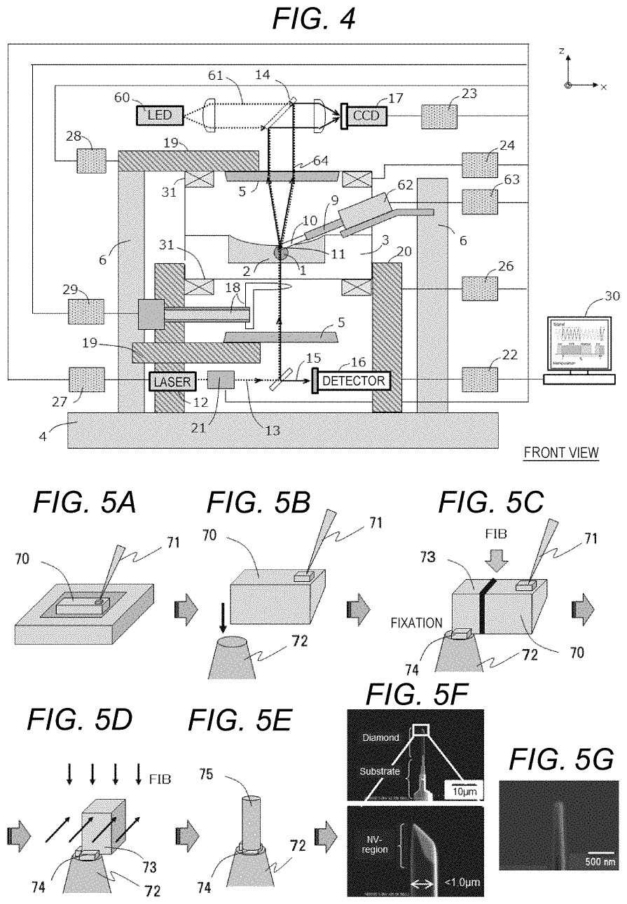

A sample measurement apparatus according to a second embodiment of the invention will be described with reference to FIG. 4. The sample measurement apparatus according to the second embodiment is a prober apparatus using the diamond including the NV center as the probe.

FIG. 4 is a front view of a basic configuration of the prober apparatus. This configuration is called an inverted configuration because the probe and the laser light approach the sample from opposite surfaces.

As illustrated in FIG. 4, the sample 1 is placed in the culture solution 2 in the sample stage 3. The diamond probe 10 is fixed on the probe base 9 made of glass or metal. Positions of the diamond probe 10 and the probe base 9 are controlled by a probe position control mechanism 62 having linear motion in an axial direction of the probe in addition to the position control in the x, y, and z directions. Since the diamond probe 10 can be linearly inserted into the sample 1, the diamond probe 10 can be easily inserted without being broken.

In the second embodiment, the microwave antenna 18 and the laser light source 12 are provided below the sample stage 3, and can approach the sample 1 from a surface opposite to the diamond probe 10 via the sample 1. Accordingly, a space between the sample and the lens 5 on the diamond probe 10 side is free, the sample environment control mechanism (see FIG. 3) such as the sample suction tube 40, the injection tube 41, the heater 46, and the flow path 48 that are provided on the side view (not shown) can be easily provided, and the sample stage 3 can be made large. That is, the sample 1 is irradiated with the laser light 13 from the laser light source 12 from the bottom surface of the sample stage 3. Therefore, the sample stage 3 is made of a material that efficiently transmits the laser light, such as a quartz glass petri dish.

The NV fluorescence excited by the sample 1 is emitted downward from a bottom surface of the sample stage 3 again, and is measured by the detector 16 provided below the sample stage 3. Therefore, the lens 5 is provided below the sample. In the second embodiment, the microwave antenna 18 is also provided under the bottom surface of the sample stage 3.

Since a distance between the microwave antenna 18 and the sample 1 is generally required to be about several tens of micrometers, the bottom surface of the sample stage 3 is assumed to be a thin quartz glass plate or the like. Since the microwave around 2.87 GHz used in the measurement of NV diamond is strongly absorbed in the solution, this method in which the microwave antenna 18 is not inserted into the culture solution has a great advantage.

In the second embodiment, an imaging system used for rough observation such as visual field search is added in an initial stage of observation in a wide visual field from an upper surface of the sample 1. That is, the sample 1 is irradiated with illumination light 61 emitted from a light source 60 such as an LED through the lens 5. Fluorescence 64 generated in the sample 1 is captured by the CCD camera 17.

Here, imaging different from that of the CCD camera 17 illustrated in the first embodiment is considered. That is, an LED light source that is less expensive than a laser is used as the light source 60, and when the green light is illuminated as in the first embodiment, only NV diamond forms a red fluorescent image. However, the light source is changed to blue light, so that NV diamond does not emit light, and an organic substance such as a cell emits the green light. An optical microscope image called a normal bright field image can be obtained by emitting white light.

In this way, by providing the irradiation light to the sample 1 in a system different from that of the laser light, it is possible to provide visual field identifying observation suitable for a structure and a composition of the sample. Accordingly, position identifying accuracy of the NV diamond is improved, and an evaluation in which identification accuracy of a cell structure is improved is achieved. This is an effect of effectively utilizing a space created by causing the apparatus configuration to be the inverted configuration.

Since the inverted configuration is a structure in which light is emitted from above and below the sample 1, the inverted configuration is particularly effective in a case of an optically transparent biological sample or an organic sample. On the other hand, it is not suitable for an evaluation of a sample having a low light transmittance such as a metal material sample or a semiconductor sample, and the configuration according to the first embodiment is effective for such a sample 1.

### Third Embodiment

A third embodiment of the invention will be described with reference to FIGS. 5A to 5G.

In the third embodiment, a microsampling method using a focused ion beam processing apparatus (hereinafter referred to as FIB) will be described with reference to FIGS. 5A to 5G as an example of processing of a probe suitable for a prober.

This processing method itself is an example of a general FIB microsampling method, and for details, refer to literatures (for example, T. Ishitani, H. Tsuboi, T. Yaguchi and H. Koike, J. Electron Microsc 43 (1994) pp. 322 to 326).

First, a region of the substrate to be used as a diamond probe later is set as a microsample region 70, a groove is formed around the microsample region 70 with an ion beam, and a microprobe 71 is brought into contact with the microsample region 70. Next, an organic tungsten gas or a phenanthrenegas flows in the FIB apparatus, and a tip of the microprobe 71 is irradiated with an ion beam or an electron beam to solidify the organic tungsten gas or the phenanthrenegas to form an adhesive, thereby fixing the microsample region 70 and the microprobe 71 (FIG. 5A).

Next, the microsample region 70 is extracted by the microprobe 71 and moved to a base material 72 (FIG. 5B). Thereafter, the organic tungsten gas or the phenanthrenegas flows again to a contact portion between the base material 72 and the microsample region 70, and the contact portion is irradiated with the ion beam, thereby forming an adhesive layer 74 and fixing the base material 72 and the microsample region 70. Subsequently, after an excess sample portion of the microsample region 70 is cut off by the FIB, fixation of the base material 72 and the microsample 73 is strengthened, and the fixation is completed (FIG. 5C).

In this way, a needle-shaped diamond probe 75 having a desired shape, for example, a diameter of 1 μm or less and a length of 20 μm or more is formed by FIB processing from various directions illustrated in FIG. 5D for the microsample 73 fixed to the base material 72 and subjected to rough processing.

Electron micrographs of a tip of the diamond probe 75 actually fabricated from the diamond substrate are illustrated in FIGS. 5F and 5G. FIG. 5F illustrates a diamond probe in which needle-shaped tungsten is used as a base material and only the tip is the NV diamond region. Here, the probe has a diameter of about 1 μm and a length of about 25 μm.

In the FIB processing illustrated in FIG. 5D, the tip is processed so as to have a knife-edge shape. By using a probe of this shape as the diamond probe 10 in FIG. 4, the diamond probe 10 can be inserted into the cell with a small resistance.

It is confirmed that, by processing the probe to be thinner in the FIB processing illustrated in FIG. 5D, the diameter of the probe can be processed to 200 nm or less as illustrated in FIG. 5G. Although the diamond probe is described above, it is needless to say that the invention can also be applied to a quantum sensing material including a similar light emitting center such as silicon carbide.

As described above, in the third embodiment, the probe has a length of 20 μm or more in the axial direction.

The probe has an axis perpendicular radius of 1 μm or less.

The axis perpendicular radius of the probe at a position of 100 nm from the tip of the probe is equal to or less than a half of the axis perpendicular radius at a position of 1 μm from the tip of the probe.

The above embodiment includes a probe made of diamond including a nitrogen-vacancy pair or silicon carbide including a silicon point defect as a base material, a microwave application mechanism, a laser light source, a photodetector, and a sample environment control mechanism.

A microscope apparatus includes a sample stage capable of holding a liquid sample, and a control mechanism that controls the laser light source and the microwave application mechanism, that analyzes a signal from the photodetector, and that displays a result of the analysis. The microscope apparatus further includes a mechanism that fixes of the sample in the sample stage and controls a position of the sample, and a substance control mechanism that can be inserted into the sample and inject or suction a substance into or from a cell.

The probe, the mechanism that fixes the sample and controls the position of the sample, and the substance control mechanism are disposed on an open surface side of the sample stage. The sample is irradiated with the laser light from a bottom surface side of the sample stage, and the obtained fluorescence is detected from the bottom surface side. The sample environment controller controls a sample temperature control mechanism, a flow rate of a solvent in the sample stage, and an additive.

According to the above embodiments, a mechanism that applies an external stimulus can be provided, and structural and electromagnetic changes of a cell due to the external stimulus can be measured with high sensitivity by the NV center.

As described above, the invention made by the inventor is specifically described based on the embodiments. However, the invention is not limited to the embodiments described above, and various modifications can be made without departing from the scope of the invention.

What is claimed is:

1. A sample measurement apparatus for measuring a state of a sample using a probe made of diamond or silicon carbide including a nitrogen-vacancy pair, the sample measurement apparatus comprising:
an environment control mechanism configured to change the state of the sample by applying an external stimulus to the sample.

2. The sample measurement apparatus according to claim 1, wherein
the environment control mechanism includes
a sample suction tube configured to determine a relative positional relation between the sample floating in a solution and the probe and to fix the sample such that the sample does not move in the solution.

3. The sample measurement apparatus according to claim 1, wherein
the environment control mechanism includes
an injection tube to be inserted into the sample and configured to inject a substance into the sample.

4. The sample measurement apparatus according to claim 1, wherein
the environment control mechanism includes
a sample suction tube configured to determine a relative positional relation between the sample floating in a solution and the probe and to fix the sample such that the sample does not move in the solution, and
an injection tube to be inserted into the sample and configured to inject a substance into the sample, and
the sample suction tube and the injection tube face each other at a predetermined angle with respect to the nitrogen-vacancy pair.

5. The sample measurement apparatus according to claim 1, further comprising:
a sample stage configured to hold the sample, wherein
the environment control mechanism includes
a heater disposed inside the sample stage and configured to adjust a temperature of the sample,
a flow path that is disposed inside the sample stage and through which a solution flows, and
a pump configured to control a flow rate of the solution flowing through the flow path.

6. The sample measurement apparatus according to claim 1, further comprising:
a sample stage configured to hold the sample, wherein
the environment control mechanism is disposed in a space formed above the sample stage.

7. The sample measurement apparatus according to claim 6, further comprising:
at least a laser light source configured to excite the nitrogen-vacancy pair, a detector, and a first lens, wherein
the laser light source, the detector, and the first lens are disposed below the sample stage.

8. The sample measurement apparatus according to claim 7, further comprising:
a microwave mechanism, wherein
the microwave mechanism is disposed below the sample stage.

9. The sample measurement apparatus according to claim 6, wherein
the sample stage is made of a material that transmits a laser light output from the laser light source, and
the sample is irradiated with the laser light from a bottom surface of the sample stage.

10. The sample measurement apparatus according to claim 7, wherein
fluorescence excited by the sample is emitted downward from a bottom surface of the sample stage and measured by the detector.

11. The sample measurement apparatus according to claim 8, wherein
the microwave mechanism is not inserted into a solution.

12. The sample measurement apparatus according to claim 6, wherein
a light source, a second lens, and a camera are disposed above the sample stage,
illumination light emitted from the light source is emitted to the sample via the second lens, and
fluorescence generated in the sample is imaged by the camera.

13. The sample measurement apparatus according to claim 1, wherein
the probe has a length of 20 μm or more in an axial direction.

14. The sample measurement apparatus according to claim 1, wherein
the probe has an axis perpendicular radius of 1 μm or less.

15. The sample measurement apparatus according to claim 1, wherein
the probe has an axis perpendicular radius at a position of 100 nm from a tip of the probe which is equal to or less than a half of an axis perpendicular radius at a position of 1 μm from the tip.

16. A sample measurement system comprising:
a control mechanism configured to analyze a measurement signal output from the sample measurement apparatus according to claim 1 and to display an analysis result.

17. An artificial insemination apparatus for measuring a reaction of a cell using a probe made of diamond or silicon carbide including a nitrogen-vacancy pair, the artificial insemination apparatus comprising:
an environment control mechanism configured to change a state of the cell by applying an external stimulus to the cell.

18. The artificial insemination apparatus according to claim 17, wherein
the environment control mechanism includes
a sample suction tube configured to determine a relative positional relation between the cell floating in a solution and the probe and to fix the cell such that the cell does not move in the solution, and
an injection tube to be inserted into the cell and configured to inject a substance into the cell, and
the sample suction tube and the injection tube face each other at a predetermined angle with respect to the nitrogen-vacancy pair.
